(19) United States
(12) Patent Application Publication
(10) Pub. No.: US 2018/0312835 A1
(43) Pub. Date: Nov. 1, 2018

(54) crRNA for detecting RSPO2 gene in body fluid with CRISPR-Cas13a specificity and applications thereof

(71) Applicant: The First Hospital of Jiaxing, Jiaxing (CN)

(72) Inventors: Ming Yao, Jiaxing (CN); Linghua Yu, Jiaxing (CN)

(21) Appl. No.: 16/010,411

(22) Filed: Jun. 16, 2018

(30) Foreign Application Priority Data
Dec. 21, 2017 (CN) 201711395369.7

(51) Int. Cl. C12N 15/11; C12N 9/22; C12Q 1/6848

(52) U.S. Cl. CPC C12N 15/11; C12N 9/22; C12N 2800/80; C12N 2310/20; C12Q 1/6848

(57) ABSTRACT

Plural crRNA for detecting RSPO2 gene in body fluid with CRISPR-Cas13a specificity and applications thereof are disclosed. The crRNA is able to construct the CRISPR-Cas13a system and specifically detect micro-RSPO2 gene in the body fluid. The present invention is non-invasive and able to test rapidly, frequently and repeatedly. Compared to the conventional liquid biopsy, the present invention detects the micro-RSPO2 in the body fluid through the fluorescence units. The present invention has the advantages of no need for high-throughput sequencing, low cost and rapid testing speed, which is able to be adopted by large scale clinical applications.

### CROSS REFERENCE OF RELATED APPLICATION

This application claims priority under 35 U.S.C. 119(a-d) to CN 201711395369.7, filed Dec. 21, 2017.

### BACKGROUND OF THE PRESENT INVENTION

### Field of Invention

The present invention relates to biotechnology, and more particularly to detecting RSPO2 gene in body fluid with CRISPR-Cas13a specificity and applications thereof.

### Description of Related Arts

Liver fibrosis is a reversible wound-healing response to a variety of insults. With chronic liver injury, this wound-healing process is presented as a progressive substitution of the functional parenchyma by scar tissue. The pathological characteristics are that various compositions, mainly collagen, of the extracellular matrix are synthesized and increased while the degradation is relatively insufficient and the interlobular septa are not formed. Further development leads to cirrhosis. The liver fibrosis is reversible. A prevention and early intervention to the liver fibrosis is the best practice to stable the condition and prevent the liver fibrosis from developing into cirrhosis and liver cancer. Conventional diagnostic golden standard for liver fibrosis is needle biopsy which is invasive testing and brings suffering and risk to the patient. Needle biopsy partially indicates the situation of the patient not integrally.

cfDNA (Cell-Free DNA) exists in peripheral blood, which is relevant to the physiological conditions and diseases. cfDNA is able to act as a biomarker to aid diagnosis. The biomarkers in the body fluid such as blood and urine are tested by liquid biopsy, which provides diagnosis assistance information. Compared to the conventional biopsy, the liquid biopsy is non-invasive and able to test frequently, repeatedly, and rapidly. Conventionally, the liquid biopsy is applied in blood test for tumor and non-invasive prenatal testing and is not for liver fibrosis diagnosis. Two problems need to be solved before applying the liquid biopsy in liver fibrosis: 1) identification of one or one set of biomarker to indicate the process of liver fibrosis; 2) specificity detecting the micro-biomarker of the liver fibrosis in the blood.

HSC (Hepatic Stellate Cell) is the primary cell type responsible for extracellular matrix synthesis and degradation. HSC activation and phenotypic switch to a myofibroblast-like cell is the central event of liver fibrogenesis. The activation of the hepatic stellate cell is regulated by multiple signal pathways. Research shows that the Wnt signal pathway affects a competence of the hepatic stellate cell and the blockage of the Wnt signal pathway suppresses the hepatic stellate cell proliferation and induces the hepatic stellate cell death. Because the Wnt signal pathway participates in various biological processes including the differentiation and maintenance of the cell form and function, immunity, and cell carcinogenesis and death, a direct blockage of the Wnt signal path may causes adverse biological effects. RSPO2 (R-spondin2) is an important newly discovered regulation factor of the Wnt signal factor, which is able to activate and enhance the Wnt/β-catenin signal pathway and play an important role in tissue differentiation, organogenesis and diseases. Detecting the RSPO2 gene in the body fluid is able to be an interim result to aid liver fibrosis diagnosis.

CRISPR-Cas (Clustered Regularly Interspaced Short Palindromic Repeats associated) widely exists in bacteria and archaea, which is a RNA-guided heritable adaptive immunity system. CRISPR (Clustered Regularly Interspaced Short Palindromic Repeats) is composed of highly conserved repeats and multiple spacers which are arranged in order. A length of the repeats is 21-48 bp. The repeats is spaced by spacers of 26-72 bp. Cas(CRISPR associated proteins) is nuclease. By choosing different Cas, CRISPR-Cas system is able to edit, suppress or activate genes, wherein Cas13a combines with crRNA to specifically identify RNA sequence.

The working principle of CRISPR-Cas13a system specifically testing RNA target sequences is as the following: 1) transcribing and processing CRISPR sequence into crRNA; 2) matching the repeats of the crRNA with the PFS (Protospacer Flanking Site) adjacent target sequence; 3) cutting the report RNA adjacent to the target RNA to release fluorophores; 4) detecting the quantity of the RNA target sequence through the fluorescence units. The CRISPR-Cas13a system is sensitive enough to detect single molecule and is able to specifically test the RSPO2 gene.

### SUMMARY OF THE PRESENT INVENTION

An object of the present invention is to specifically test the RSPO2 gene in the body fluid by adopting CRISPR-Cas13a system. The present invention extracts DNA from the body fluid, amplifies RSPO2 gene and specifically test the RSPO2 gene in the body fluid.

The technical solution of the present invention is as follow.

Firstly, the present invention provides plural crRNA of a specifically targeted human RSPO2 gene in a CRISPR-Cas13a system, wherein a crRNA sequence format is: 5′—a direct repeat combined with a Cas13a protein-crRNA spacer-3; the crRNA spacer is as any one sequence of SEQ ID NO: 2, 6, 10, 14.

Optionally, target sequences of four crRNA is as any one sequence of SEQ ID NO: 1, 5, 9, 13; corresponding spacers sequence is as SEQ ID NO 2, 6, 10, 14 respectively; corresponding plural PFS are C, U, C, A (the corresponding spacer and the corresponding PFS of the target sequence as SEQ ID NO 1 is as SEQ ID NO: 2 and C respectively. Similar rules are able to be applied on the remaining three crRNA).

Optionally, a Cas13a protein is LwCas13a. The direct repeat in the crRNA adopts the direct repeat GAUUUAGACUACCCCAAAAACGAAGGGGACUAAAAC which combined with the LwCas13a. The four crRNA sequences are as any one sequence of SEQ ID NO: 3, 7, 11, 15.

Optionally, a Cas13a protein is LshCas13a. The direct repeat in the crRNA adopts the direct repeat CCACCCCAAUAUCGAAGGGGACUAAAAC which combined with the LshCas13a. The four crRNA sequences are as any one sequence of SEQ ID NO: 4, 8, 12, 16.

Secondly, a CRISPR-Cas13a system is constructed with the crRNA mentioned in the above mentioned optional optimizations.

Optionally, the CRISPR-Cas13a system further comprises plasmids which are able to express a Cas13a protein, a RNA reporter and a nuclease buffer solution. Other necessary reagents and compositions are included based on requirements for constructing CRISPR-Cas13a system. The sample RNA under test is added into the CRISPR-Cas13a system. The quantity of the RNA target sequence is achieved through fluorescence analysis and the RSPO2 gene expression is thus shown.

Thirdly, the present invention provides an application of the above mentioned CRISPR-Cas13a system in a human RSPO2 gene test.

Fourthly, the present invention provides a method of a RSPO2 gene liquid biopsy based on a CRISPR-Cas13a. The method comprises steps as follow: centrifugally separating peripheral blood; extracting a sample DNA from a supernatant; adding a T7 promoter sequence to a 5′ end of a DNA strand waiting for transcription of the sample DNA; amplifying RSPO2 genes in the sample DNA by PCR (polymerase chain reaction); generating a sample RNA with a PCR products containing T7 promoter under a help of T7 RNA polymerase; extracting and purifying; incubating the crRNA mentioned above, the sample RNA, plasmids which are able to express a Cas13a protein and a RNA reporter in a nuclease buffer solution; and analyzing a quantity of a RNA target sequence expressed by the RSPO2 gene through fluorescence units.

The fourth and fifth mentioned part provides new technical means for RSPO2 gene biopsy in the body fluid. The applications and methods are able to be applied in non-diagnostic purposes, such as commercial testing, scientific research or preparation of test kits. The test results aid the clinical liver fibrosis diagnosis.

Fifthly, the present invention provides an application of the crRNA mentioned above in preparing kits for testing human RSPO2 gene. crRNA is able to be prepared as kits by ligation for commercial promotion.

The benefits of the present invention are as follow: the present invention discloses a method for detecting RSPO2 gene in the body fluid by adopting CRISPR-Cas13a system. The micro-RSPO2 gene is specifically tested in the body fluid, which is an interim result to aid liver fibrosis diagnosis. The present invention is non-invasive and able to test rapidly, frequently and repeatedly. The present invention is capable of providing diagnosis assistance information for prevention and early intervention of the liver fibrosis. Compared to the conventional liquid biopsy, the present invention detects the micro-RSPO2 in the body fluid through the fluorescence units. The present invention has the advantages of no need for high-throughput sequencing, low cost and rapid testing speed, which is able to be adopted by large scale clinical applications.

### BRIEF DESCRIPTION OF THE DRAWINGS

FIG. 1 illustrates a working principle of RSPO2 gene liquid biopsy based on CRISPR-Cas13a;

FIG. 2 is a perspective view of a structure of the CRISPR-Cas13a;

FIG. 3 is a result of crRNA testing the target RNA, which is designed for 1, 2, 3, 4 target of the RSPO2 gene;

FIG. 4 is a testing result of RSPO2 in sample peripheral blood, which adopts CRISPR-Cas13a system.

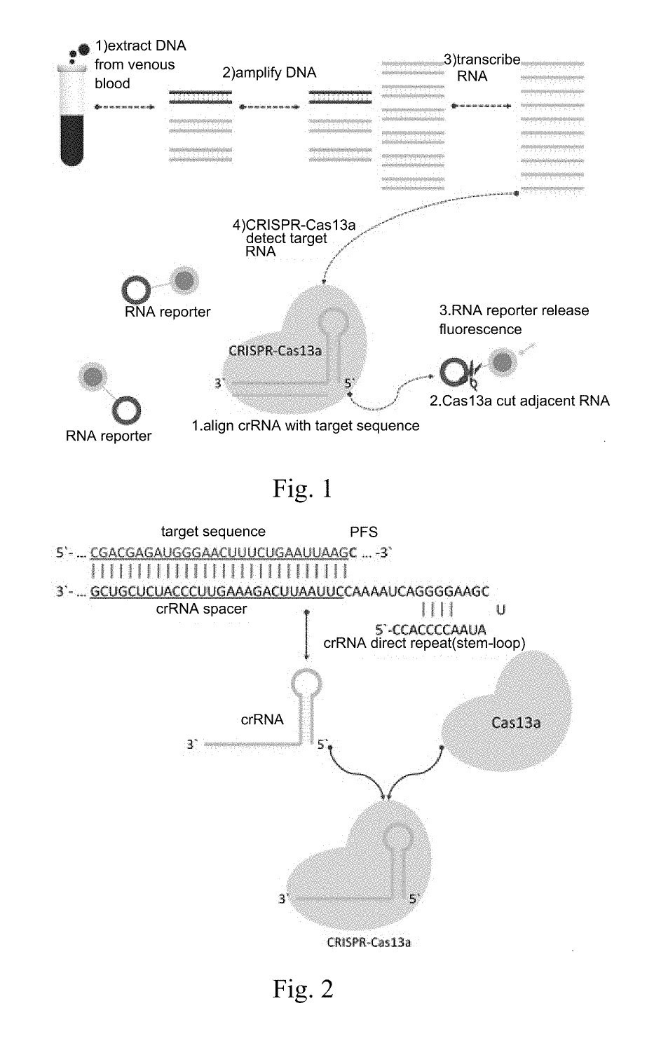

### DETAILED DESCRIPTION OF THE PREFERRED EMBODIMENT

Referring the drawings, according to preferred embodiments, the present invention is further illustrated. The technical features described in the embodiments of the present invention are able to be recombined when there are no conflicts.

The method for constructing the CRISPR-Cas13a of the specified target human RSPO2 gene in the present invention comprises the following steps.

1. Designing the crRNA of the specified target human RSPO2, which further comprises the following sub-steps;

1) designing the crRNA of the target human RSPO2 gene;

wherein the CRISPR-Cas13a is classified in CRISPR Class 2 type VI system;

there is no need for tracrRNA (trans-activating crRNA) to intervene the mature of crRNA and the binding with Cas13a; the design of crRNA is different to the design of sgRNA in the CRISPR-Cas9; there is no specified design principles for crRNA in the CRISPR-Cas13a system; the design principles for crRNA of the target human RSPO2 gene, which are based on the experiences accumulated in the previous work, are as follow:

a) crRNA comprises spacer and DR (direct repeat), wherein the format is 5′-a direct repeat combined with a Cas13a protein-crRNA spacer-3; the direct repeat is identified by Cas13a to guarantee a match and combination with a selected Cas13a.

b) a length of the crRNA spacer is 22-28 nucleotide sequences;

c) a target of the crRNA spacer on the RSPO2 gene is in exon;

d) a target sequence 3′ end PFS (protospacer flanking site) is not G, which matches the crRNA spacer;

e) the crRNA direct repeat is longer than 24 nucleotide sequences;

f) the crRNA direct repeat comprises stem loop structures;

g) the crRNA spacer is mediated by seed region, which must not mismatch the target sequence while combining;

2) selecting the crRNA of the targeted human RSPO2 gene;

a) ensuring the unique of crRNA target sequence and ensuring the crRNA target sequence does not homologous with a gene sequence other than the human RSPO2 gene by adopting BLAST in the NCBI database;

b) the crRNA target must not be too close to a start codon ATG;

c) low Off-Target rate;

crRNA of plural targeted human RSPO2 gene is thus designed; wherein the target sequence of the RSPO2 gene corresponding to different locus is illustrated in table 1;

TABLE 1
the target sequence of the RSPO2 gene
corresponding to different locus

| NO | Target sequence | PFS | crRNA spacer |
|---|---|---|---|
| 1 | 5′- | C | 5′- |
| | CGACGAGAUGGGAACUUU | | CUUAAUUCAGAAAGUUCC |
| | CUGAAUUAAG-3′ | | CAUCUCGUCG-3′ |
| 2 | 5′- | U | 5′- |
| | GCAAUUCCCGCGCUGGUU | | CUCCCCAGAAAACCAGCGC |
| | UUCUGGGGAG-3′ | | GGGAAUUGC -3′ |
| 3 | 5′- | C | 5′- |
| | GUUUUCUGGGGAGUCCUC | | CUCUGGAGGCGAGGACUCC |
| | GCCUCCAGAG -3′ | | CCAGAAAAC-3′ |
| 4 | 5′- | A | 5′- |
| | GAGUCCUCGCCUCCAGAG | | ACAUAACUAGCUCUGGAG |
| | CUAGUUAUGU-3′ | | GCGAGGACUC-3′ |

2. synthesizing the crRNA, which further comprises the following sub-steps:

1) the synthesizing of crRNA;

a) adding GAUUUAGACUACCCCAAAAACGAAGGGGACUAAAAC (the direct repeat corresponding to LwCas13a protein) or CCACCCCAAUAUCGAAGGGGACUAAAAC (the direct repeat corresponding to LshCas13a protein) to 5′ end according to the selected crRNA spacer to achieve crRNA sequence; wherein

b) the format of the crRNA sequence is 5′-GAUUUAGACUACCCCAAAAACGAAGGGGACUAAAAC-crRNA spacer-3′; or 5′-CCACCCCAAUAUCGAAGGGGACUAAAAC-crRNA spacer-3′;

c) synthesizing DNA according to the crRNA and adding T7 promoter sequence (5′-TAATACGACTCACTATAGGG-3′) to 5′;

d) generating RNA with DNA containing T7 promoter under a help of T7 RNA polymerase; extracting and purifying; achieving sufficient crRNA;

2) synthesizing the target RNA sequence;

a) synthesizing the corresponding DNA according to the target RNA sequence; adding T7 promoter sequence (5′-TAATACGACTCACTATAGGG-3′) to 5′;

wherein the target RNA 1-4 sequence is illustrated in Table 2;

TABLE 2
target RNA sequence

| No. | Target RNA sequence |
|---|---|
| 1 | CGACGAGAUGGGAACUUUCUGAAUUAAGCAGCAAUUCCC |
| | GCGCUGGUUUUCUGGGGAGUCCUCGCCUCCAGAGCUAGU |
| | UAUGUAUCAAAUCCCAUUUGCAAGGGUUGUUUGUCUUGU |
| | UCAAAGGACAAUGGGUGUAGCCGAUGUCAACAGAAG |
| 2 | GCAGCAAUUCCCGCGCUGGUUUUCUGGGGAGUCCUCGCCU |
| | CCAGAGCUAGUUAUGUAUCAAAUCCCAUUUGCAAGGGUU |
| | GUUUGUCUUGUUCAAAGGACAAUGGGUGUAGCCGAUGUC |
| | AACAGAAGUUGUUCUUCUUCCUUCGAAGAGAAGGG |
| 3 | GCUGGUUUUCUGGGGAGUCCUCGCCUCCAGAGCUAGUUA |
| | UGUAUCAAAUCCCAUUUGCAAGGGUUGUUUGUCUUGUUC |
| | AAAGGACAAUGGGUGUAGCCGAUGUCAACAGAAGUUGUU |
| | CUUCUUCCUUCGAAGAGAAGGGAUGCGCCAGUAUGG |
| 4 | GGGAGUCCUCGCCUCCAGAGCUAGUUAUGUAUCAAAUCC |
| | CAUUUGCAAGGGUUGUUUGUCUUGUUCAAAGGACAAUGG |
| | GUGUAGCCGAUGUCAACAGAAGUUGUUCUUCUUCCUUCG |
| | AAGAGAAGGGAUGCGCCAGUAUGGAGAGUGCCUGC |

b) generating a sample RNA with DNA containing T7 promoter under a help of T7 RNA polymerase; extracting and purifying; achieving target RNA;

3) validating the crRNA;

a) incubating the target RNA, crRNA, plasmids which are able to express a Cas13a protein and a RNA reporter (RNA Alert V2, Thermo Scientific) with fluorophore in a nuclease buffer solution for 1 to 3 hours; wherein the plasmids matches the crRNA; when the direct repeat in the crRNA adopts a direct repeat which is able to combine with LwCas13a protein, the plasmids adopts Twinstrep-SUMO-huLwCas13a (Feng Zhang, Science 2017) which contains LwCas13a; similar rules are applied on the direct repeat corresponding to the LshCas13a protein;

b) detecting the fluorescence units by a fluorescence reader;

3. testing the RSPO2 gene in the peripheral blood by the CRISPR-Cas13a system; which further comprises the following sub-steps;

1) extracting the sample DNA; wherein

the peripheral blood is centrifugally separated into blood plasma and blood cell; a sample DNA is extracted from a supernatant;

2) amplifying RSPO2 genes in the sample DNA; wherein

the T7 promoter sequence (TAATACGACTCACTATAGGG) is added to the 5′ end of the upstream primer of the sample DNA strand waiting for transcription; the RSPO2 gene in the sample DNA is amplified by PCR (polymerase chain reaction);

3) generating RNA by vitro transcription; wherein

RNA is generated with a PCR products containing T7 promoter under a help of T7 RNA polymerase; extracting and purifying the gel;

4) detecting RSPO2 gene based on the CRISPR-Cas13a system; wherein

incubating the constructed crRNA of the targeted human RSPO2 gene, the RNA generated by sample DNA transcription, plasmids which are able to express a corresponding Cas13a protein and a RNA reporter (RNA Alert V2, Thermo Scientific) in a nuclease buffer solution for 1 to 3 hours at 37° C.; a quantity of a RNA target sequence expressed by the RSPO2 gene is analyzed through fluorescence units;

the plural crRNA provided in the present invention are able to be adopted combinedly, wherein any two or plural crRNA are able to be adopted combinedly to detect plural target.

The following embodiments further explain the present invention. The embodiments are not independent but a consecutive process. The embodiments are for illustrating the present invention, which are not a limitation to the present invention. Unless otherwise stated, the molecular biotechnology involved in the embodiments, such as the vitro transcription, the PCR amplification and the fluorescence units, is regular technology which is understood by one skilled in the art; the instruments, reagents, plasmids, cell strains and etc. are available from the market by a skilled in the art.

### Embodiment 1 Designing the crRNA Sequence

the design of crRNA of CRISPR-Cas13a is different to the design of sgRNA in the CRISPR-Cas9; there is no specified design principles for crRNA in the CRISPR-Cas13a system; the design principles for crRNA of the target human RSPO2 gene, which are based on the experiences accumulated in the previous work, are as follow: a) crRNA comprises spacer and DR; b) a length of the crRNA spacer is 22-28 nucleotide sequences; c) a target of the crRNA spacer on the RSPO2 gene is in exon; d) a target sequence 3′ end PFS (protospacer flanking site) is not G; e) the crRNA spacer is mediated by seed region, which must not mismatch the target sequence while combining; f) the crRNA direct repeat is longer than 24 nucleotide sequences; g) the crRNA direct repeat comprises stem loop structures. The crRNA in the present embodiment is based on two Cas13a proteins which are LshCas13a and LwCas13a. The stem-loop structure is as follow:

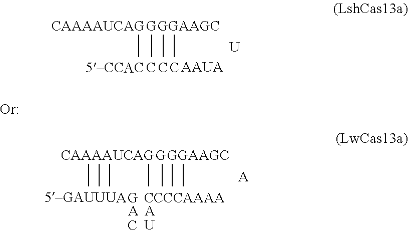

Based on the principles, the present invention designs candidate crRNA sequences of plural targeted human RSPO2 gene for selection.

### Embodiment 2 the Selecting of the crRNA Sequence

Ensuring the unique of crRNA target sequence and ensuring the crRNA target sequence does not homologous with a gene sequence other than the human RSPO2 gene by adopting BLAST (www.ncbi.nlm.nig.gov/Blast) to carry out homological analysis between the candidate crRNA sequences and the genome database; wherein the crRNA sequence which is able to efficiently and specifically test the human RSPO2 gene is selected according to the following principles: a) the crRNA target must not be too close to a start codon ATG; b) low Off-Target rate.

crRNA spacer corresponding to four targeted human RSPO2 gene of different locus is selected according to the principles. The four target sequences are as SEQ ID NO. 1, 5, 9, 13. The crRNA spacer corresponding to the target sequences are as SEQ ID NO. 2, 6, 10, 14. The correspondence among the target sequence, crRNA spacer and the PFS are illustrated in the table 1, which is no need for further explanation. crRNA sequences of eight targeted human RSPO2 gene are designed in the present embodiment to corresponds two type of Cas13a proteins; wherein SEQ ID NO. 3, 7, 11, 15 are crRNA sequences for LwCas13a; SEQ ID NO. 4, 8, 12, 16 are crRNA sequences for LshCas13a.

### Embodiment 3 Synthesizing DNA by crRNA

The crRNA is synthesized into DNA and is vitro transcribed to RNA when in use in the present invention for the convenience of storage and amplification in the following experiments; wherein 1) achieving crRNA sequences by adding GAUUUAGACUACCCCAAAAACGAAGGGGACUAAAAC (the direct repeat corresponding to the LwCas13a protein) or CCACCCCAAUAUCGAAGGGGACUAAAAC (the direct repeat corresponding to the LshCas13a protein) to 5′ end according to the selected crRNA spacer; 2) the format of the crRNA sequence is

5′-GAUUUAGACUACCCCAAAAACGAAGGGGACUAAAAC-crRNA spacer-3′ (LwCas13a) or 5′-CCACCCCAAUAUCGAAGGGGACUAAAAC-crRNA spacer-3′; (LshCas13a); 3) adding T7 promoter sequence (TAATACGACTCACTATAGGG) to 5′; wherein the format of the DNA sequence is as follow:

forward sequence (LwCas13a):
5′- TAATACGACTCACTATAGGG - GATTTAGACTACCCCAAAAACGA
AGGGGACTAAAAC - DNA sequence corresponding to
crRNA spacer-3′;
reverse sequence (LwCas13a):
5′- DNA sequence corresponding to crRNA spacer -
GTTTTAGTCCCCTTCGTTTTTGGGGTAGTCTAAATC -
CCCTATAGTGAGTCGTATTA -3′;
forward sequence (LshCas13a):
5′ -TAATACGACTCACTATAGGG - CCACCCCAATATCGAAGGGGACT
AAAAC - DNA sequence corresponding to crRNA
spacer -3′;
reverse sequence (LshCas13a):
5′ - DNA sequence corresponding to crRNA spacer -
GTTTTAGTCCCCTTCGATATTGGGGTGG- CCCTATAGTGAGTCGTATT
A -3′;

4) taking LwCas13a for example, the dsDNA sequence is achieved by mutating and annealing the synthesized forward oligo and reverse oligo; wherein the dsDNA sequence is listed in table 3.

TABLE 3
the DNA sequence corresponding to crRNA

| No. | DNA sequence |
|---|---|
| Forward | 5′-TAATACGACTCACTATAGGGGATTTAGACTACCCCAA |
| 1 | AAACGAAGGGGACTAAAACCGACGAGATGGGAACTTTCTG |
| | AATTAAG -3′ |
| Reverse | 5′-CTTAATTCAGAAAATTCCCATCTCGTCGGTTTTAGTC |
| 1 | CCCTTCGTTTTTGGGGTAGTCTAAATCCCCTATAGTGAGT |
| | CGTATTA -3′ |
| Forward | 5′-TAATACGACTCACTATAGGGGATTTAGACTACCCCAA |
| 2 | AAACGAAGGGGACTAAAACGCAATTCCCGCGCTGGTTTTC |
| | TGGGGAG -3′ |
| Reverse | 5′-CTCCCCAGAAAACCAGCGCGGGAATGCGTTTTAGTCC |
| 2 | CCTTCGTTTTTGGGGTAGTCTAAATCCCCTATAGTGAGTC |
| | GTATTA -3′ |
| Forward | 5′-TAATACGACTCACTATAGGGGATTTAGACTACCCCAA |
| 3 | AAACGAAGGGGACTAAAACGTTTTCTGGGGAGTCCTCGCC |
| | TCCAGAG -3′ |
| Reverse | 5′-CTCTGGAGGCGAGGACTCCCCAGAAAACGTTTTAGTC |
| 3 | CCCTTCGTTTTTGGGGTAGTCTAAATCCCCTATAGTGAGT |
| | CGTATTA -3′ |
| Forward | 5′-TAATACGACTCACTATAGGGGATTTAGACTACCCCAA |
| 4 | AAACGAAGGGGACTAAAACGAGTCCTCGCCTCCAGAGCTA |
| | GTTATGT -3′ |
| Reverse | 5′-ACATAACTAGCTCTGGAGGCGAGGACTCGTTTTAGTC |
| 4 | CCCTTCGTTTTTGGGGTAGTCTAAATCCCCTATAGTGAGT |
| | CGTATTA -3′ |

### Embodiment 4 Amplifying the Target DNA Sequence by PCR

1) adding T7 promoter sequence (TAATACGACTCACTATAGGG) to the primer 5′ end; the primers of different target sequence 1 to 4 (the target RNA sequence needs to be pre-synthesized into the corresponding DNA) and the RSPO2 gene are listed in table 4, which are able to be selected according to the PCR amplified sequence;

TABLE 4
the PCR primer adopted by different amplified
sequences

| Target | | |
|---|---|---|
| sequence | Forward primer | Reverse primer |
| Target | 5′-CGACGAGATGGGAACT | 5′-CTTCTGTTGACATCGG |
| sequence | TTCTG-3′ | CTACA-3′ |
| 1 | | |
| Target | 5′-GCAGCAATTCCCGCGC | 5′-CCCTTCTCTTCGAAGG |
| sequence | TGGTT-3′ | AAGAA-3′ |
| 2 | | |
| Target | 5′-GCTGGTTTTCTGGGGA | 5′-CCATACTGGCGCATCC |
| sequence | GTCCT-3′ | CTT-3′ |
| 3 | | |
| Target | 5′-GGGAGTCCTCGCCTCC | 5′-GCAGGCACTCTCCATA |
| sequence | AGA-3′ | CTGGC-3′ |
| 4 | | |
| RSPO2 | 5′-GTTTCCTCAGGGCATT | 5′-TGCATTATTTCCCTGG |
| | GCTT-3′ | CTGA-3′ |

2) the PCR reaction system is as the following:

| | |
|---|---|
| 10 × buffer | 2 μl |
| dNTP (10 mM respectively) | 0.4 μl |
| forward primer (20 μM) | 0.4 μl |
| reverse primer (20 μM) | 0.4 μl |
| template DNA (1 μg) | 0.2 μl |
| Taq | 0.4 μl |
| DEPC water | 16.2 μl |

3) PCR conditions: 94° C. for 5 minutes, 1 cycle; 94° C. for 30 seconds, 56° C. for 30 seconds, 72° C. for 30 seconds, altogether 35 cycles; 72° C. for further 5 minutes.

### Embodiment 5 Vitro Transcribing the RNA

1) shaking even and pulse centrifuging the solubilized reagent; collecting and placing all the compositions on ice;

2) the reaction system for the vitro transcription is as follow:

| | |
|---|---|
| Nuclease-free water | 13 μl |
| 10 × Reaction Buffer | 2 μl |
| dNTP (10 mM respectively) | 2 μl |
| template DNA | 1 μg |
| T7 RNA Polymerase Mix | 2 μl |

3) mixing even, pulse centrifuging and collecting the mixed solution; incubating for 2 hours at 37° C.;

4) adding 1 μg Dnase I in the reaction system; incubating for 15 minutes at 37° C. to digest the template DNA; and

5) extracting and purifying the synthesized RNA.

### Embodiment 6 Validating the crRNA

1) amplifying the target sequence 1 to 4 listed in the table 1 by PCR according to the embodiment 4;

2) vitro transcribing the target sequences to RNA (target RNA 1 to 4) and vitro transcribing the DNA corresponding to the crRNA to crRNA according to the embodiment 5;

3) taking LwCas13a for example, constructing the CRISPR-Cas13a system by adopting the target RNA and the corresponding crRNA:

| | |
|---|---|
| Target RNA (30 nM) | 2 μl |
| crRNA (22.5 nM) | 2 μl |
| Twinstrep-SUMO-huLwCas13a (45 nM) | 2 μl |
| RNA se Alert v2 (125 nM) | 10 μl |
| RNase inhibitor | 2 μl |
| nuclease assay buffer | 32 μl |

wherein the nuclease assay buffer comprises 40 mM Tris-HCL, 60 mM NaCl, 6 mM MgCl₂; the PH value of the nuclease assay buffer is 7.3;

4) incubating for 1 to 3 hours at 37° C.; and

5) reading by the fluorescence reader with an interval of 5 minutes.

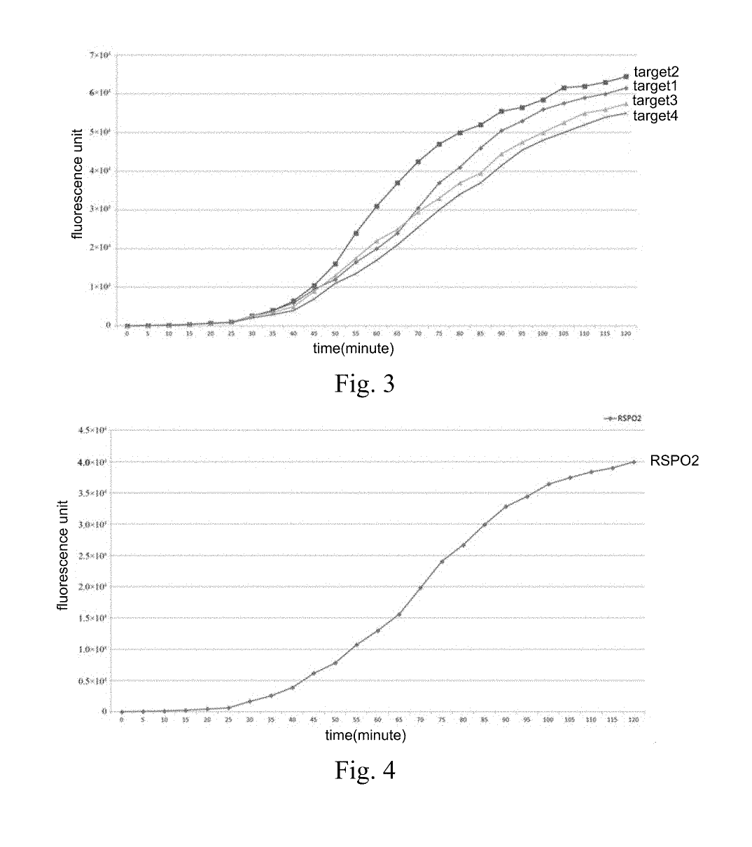

The Reporter RNA releases the fluorophore when the CRISPR-Cas13a targeted human RSPO2 gene combines with the target sequence and the Cas13a protein cuts the Reporter RNA near the target RNA; the quantity of the RNA target sequence is detected by the fluorescence units. As illustrated in the FIG. 3, the crRNA corresponding to the RSPO2 target 1, 2, 3, 4 combines with the corresponding RSPO2 RNA target sequence and the fluorescence units rises significantly after 30 minutes. The crRNA sequence designed by the present invention is validated.

### Embodiment 7 Extracting the cfDNA from the Sample Blood

1) separating the blood

centrifuging the blood sample (4° C., 1600 g, 10 minutes); re-centrifuging the supernatant (4° C., 1600 g, 10 minutes); collecting the supernatant;

2) lysis

the reaction system is as follow:

| | |
|---|---|
| supernatant in 1) | 1 ml |
| Proteinase K | 100 μl |
| ACL buffer | 2 ml |

mixing even in the centrifuge tube and incubating for 30 minutes at 60° C.;

adding 1.8 ml buffer ACB, mixing even and incubating on ice for 5 minutes;

3) column chromatography

adding 1 ml the mixed solution to QIAamp Mini column and centrifuging (8000 g, 1 minute); and

4) washing

a) adding ACW1 600 μl, ACW2 750 μl and absolute ethyl alcohol 750 μl in sequence in the spin column; centrifuging every time before adding the next solution (8000 g, 1 minute); discarding the supernatant; b) centrifuging the spin column inside the 2 ml collection tube (2000 g, 3 minutes); c) eluting the cfDNA, centrifuging (200 g, 1 minute) and collecting the cfDNA.

5) purifying the cfDNA

a) adding 25 μl Agencour AMPure XP in the centrifuge tube contains cfDNA, mixing even, incubating for 5 minutes at room temperature, placing the solution on a magnetic separator and transferring the supernatant to the centrifuge tube after the solution is clear; b) adding 48 μl Agencour AMPure XP, incubating for 5 minutes at room temperature, placing the solution on a magnetic separator and discarding the supernatant after the solution is clear; c) washing the magnetic beads with 70% ethanol, centrifuging (8000 g, 1 minute), absorbing the remnant liquid and drying for 5 minutes at room temperature; and d) eluting the magnetic beads with 20 μl water to achieve the purified cfDNA.

### Embodiment 8 Detecting the RSPO2 Gene in the Sample Blood by CRISPR-Cas13a

Taking the crRNA sequence 1 (SEQ ID NO. 3) for example, the CRISPR-Cast 3a detects the RSPO2 gene in the sample blood, which comprises the following steps:

1) extracting the sample cfDNA

extracting the cfDNA from the sample blood according to the embodiment 7;

2) amplifying the RSPO2 gene in the sample under test

adding T7 promoter sequence (TAATACGACTCACTATAGGG) to the upstream primer 5′ and amplifying the RSPO2 gene in the sample DNA by PCR according to the embodiment 4;

3) generating RNA by vitro transcription

generating the RNA with a PCR products containing T7 promoter under a help of T7 RNA polymerase and extracting and purifying the gel according to the embodiment 5;

4) detecting the RSPO2 gene by the CRISPR-Cas13a system

detecting the RSPO2 gene and constructing the CRISPR-Cas13a system according to the embodiment 6:

| | |
|---|---|
| sample RNA (30 nM) | 2 μl |
| crRNA (22.5 nM) | 2 μl |
| Twinstrep-SUMO-huLwCas13a (45 nM) | 2 μl |
| RNA se Alert v2 (125 nM) | 10 μl |
| RNase inhibitor | 2 μl |
| nuclease assay buffer | 32 μl |

incubating for 1 to 3 hours at 37° C.; reading by the fluorescence reader with an interval of 5 minutes.

The Reporter RNA releases the fluorophore when the CRISPR-Cas13a targeted RSPO2 gene combines with the target sequence and the Cas13a protein cuts the Reporter

RNA near the target RNA; the quantity of the RNA target sequence is detected by the fluorescence units. As illustrated in the FIG. 4, the fluorescence units rises significantly after 30 minutes, which proves the CRISPR-Cas13a system is able to specifically detect the RSPO2 in the sample blood.

The embodiments are just preferred solution for the present invention, which are not a limitation to the present invention. One skilled in the art is able to alter and modify the embodiments within the spirit and range of the present invention. Any technical solution which adopts equivalent replacements alterations is within the protection range of the present invention.

What is claimed is:

1. Plural crRNA of a specifically targeted human RSPO2 gene in a CRISPR-Cas13a system, wherein crRNA sequences format is: 5′-a direct repeat combined with a Cas13a protein-crRNA spacer-3; the crRNA spacer is as any one sequence of SEQ ID NO: 2, 6, 10, 14.

2. The plural crRNA of the specifically targeted human RSPO2 gene in the CRISPR-Cas13a system, as recited in claim 1, wherein target sequences of four crRNA are as any one sequence of SEQ ID NO: 1, 5, 9, 13; corresponding spacers is as SEQ ID NO 2, 6, 10, 14 respectively; corresponding plural PFS are C, U, C, A.

3. The plural crRNA of the specifically targeted human RSPO2 gene in the CRISPR-Cas13a system, as recited in claim 1, wherein a Cas13a protein is LwCas13a, crRNA sequences are as any one sequence of SEQ ID NO: 3, 7, 11, 15.

4. The plural crRNA of the specifically targeted human RSPO2 gene in the CRISPR-Cas13a system, as recited in claim 1, wherein the Cas13a protein is LshCas13a, the crRNA sequences are as any one sequence of SEQ ID NO: 4, 8, 12, 16.

5. A method of a RSPO2 gene liquid biopsy based on a CRISPR-Cas13a system which constructed with the crRNA, as recited in claim 1 comprising steps as follow: centrifugally separating peripheral blood; extracting a sample DNA from a supernatant; adding a T7 promoter sequence to a 5′ end of a sense primer of a DNA strand waiting for transcription of the sample DNA; amplifying RSPO2 genes in the sample DNA by PCR (polymerase chain reaction); generating a sample RNA from a PCR products containing T7 promoter with T7 RNA polymerase; extracting and purifying; incubating the crRNA, the sample RNA, plasmids to express a Cas13a protein and a RNA reporter in a nuclease buffer solution; and analyzing a quantity of a RNA target sequence expressed by the RSPO2 gene through fluorescence units.

6. The method of a RSPO2 gene liquid biopsy based on a CRISPR-Cas13a system, as recited in claim 5, wherein the CRISPR-Cas13a system further comprising plasmids to express a Cas13a protein, a RNA reporter and a nuclease buffer solution.

7. Kits for testing human RSPO2 gene, comprising the crRNA as recited in claim 1.


SEQUENCE LISTING (16 sequences)

SEQ ID NO: 1 - RNA, 28 nt, Artificial sequences
  RSPO2 target 1 oligo sequence
cgacgagaugggaacuuucugaauuaag

SEQ ID NO: 2 - RNA, 28 nt, Artificial sequences
  crRNA spacer of targeted RSPO2 target 1
cuuaauucagaaaguucccaucucgucg

SEQ ID NO: 3 - RNA, 64 nt, Artificial sequences
  crRNA sequence(LwCas13a) of targeted RSPO2 target 1
gauuuagacuaccccaaaaacgaaggggacuaaaaccuuaauucagaaaguucccaucucgucg

SEQ ID NO: 4 - RNA, 56 nt, Artificial sequences
  crRNA sequence(LshCas13a) of targeted RSPO2 target 1
ccaccccaauaucgaaggggacuaaaaccuuaauucagaaaguucccaucucgucg

SEQ ID NO: 5 - RNA, 28 nt, Artificial sequences
  RSPO2 target 2 oligo sequence RSPO2
gcaauucccgcgcugguuuucuggggag

SEQ ID NO: 6 - RNA, 28 nt, Artificial sequences
  crRNA spacer of targeted RSPO2 target 2
cuccccagaaaaccagcgcgggaauugc

SEQ ID NO: 7 - RNA, 64 nt, Artificial sequences
  crRNA sequence(LwCas13a) of targeted RSPO2 target 2
gauuuagacuaccccaaaaacgaaggggacuaaaaccuccccagaaaaccagcgcgggaauugc

SEQ ID NO: 8 - DNA, 56 bp, Artificial sequences
  crRNA sequence(LshCas13a) of targeted RSPO2 target 2
ccaccccaauaucgaaggggacuaaaaccuccccagaaaaccagcgcgggaauugc

SEQ ID NO: 9 - RNA, 28 nt, Artificial sequences
  RSPO2 target 3 oligo sequence RSPO2
guuuucuggggaguccucgccuccagag

SEQ ID NO: 10 - RNA, 28 nt, Artificial sequence
  crRNA spacer of targeted RSPO2 target 3
cucuggaggcgaggacuccccagaaaac

SEQ ID NO: 11 - RNA, 64 nt, Artificial sequence
  crRNA sequence (LwCas13a) of targeted RSPO2 target 3
gauuuagacuaccccaaaaacgaaggggacuaaaaccucuggaggcgaggacuccccagaaaac

SEQ ID NO: 12 - RNA, 56 nt, Artificial sequence
  crRNA sequence (LshCas13a) of targeted RSPO2 target 3
ccaccccaauaucgaaggggacuaaaaccucuggaggcgaggacuccccagaaaac

SEQ ID NO: 13 - RNA, 28 nt, Artificial sequence
  RSPO2 target 4 oligo sequence RSPO2RSPO2
gaguccucgccuccagagcuaguuaugu

SEQ ID NO: 14 - RNA, 28 nt, Artificial sequence
  crRNA spacer of targeted RSPO2 target 4
acauaacuagcucuggaggcgaggacuc

SEQ ID NO: 15 - RNA, 64 nt, Artificial sequence
  crRNA sequence (LwCas13a) of targeted RSPO2 target 4
gauuuagacuaccccaaaaacgaaggggacuaaaacacauaacuagcucuggaggcgaggacuc

SEQ ID NO: 16 - RNA, 56 nt, Artificial sequence
  crRNA sequence (LshCas13a) of targeted RSPO2 target 4
ccaccccaauaucgaaggggacuaaaacacauaacuagcucuggaggcgaggacuc